(19) United States
(12) Patent Application Publication
(10) Pub. No.: US 2019/0256902 A1
(43) Pub. Date: Aug. 22, 2019

(54) GENE SEQUENCING CHIP, DEVICE AND METHOD

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Peizhi CAI, Beijing (CN); Fengchun PANG, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 15/776,224

(22) PCT Filed: Oct. 24, 2017
(86) PCT No.: PCT/CN2017/107437

§ 371 Date: May 15, 2018

(30) Foreign Application Priority Data
Apr. 28, 2017 (CN) 201710293304.5

(51) Int. Cl. C12Q 1/6869; G02F 1/1335; G02F 1/1339; G01J 4/04

(52) U.S. Cl. CPC C12Q 1/6869; G02F 1/133528; G01J 2004/001; G01J 4/04; G02F 1/1339

(57) ABSTRACT

A gene sequencing chip, a gene sequencing device and a gene sequencing method are provided. The gene sequencing chip includes a first substrate, a common electrode, a second substrate and a liquid crystal layer. The first substrate is opposite to the second substrate, the liquid crystal layer is disposed between the first substrate and the second substrate. A side of the second substrate away from the first substrate includes at least one groove hollowed in the second substrate, the at least one groove is configured to contain a sample to be test, a bottom of the at least one groove close to the first substrate is provided with an ion sensitive film, the ion sensitive film is configured to sense an ion generated by a gene sequencing reaction within the groove to generate a voltage and generate an electric field with the common electrode.

### CROSS REFERENCE

The present application claims priority of China Patent application No. 201710293304.5 filed on Apr. 28, 2017, the content of which is incorporated in its entirety as portion of the present application by reference herein.

### TECHNICAL FIELD

Embodiments of the present disclosure relate to a gene sequencing chip, a gene sequencing device and a gene sequencing method.

### BACKGROUND

With the continuous development of gene sequencing technology, the gene sequencing technology has gradually become the most commonly used technology in the modern molecular biology research and has a broad application scene. Therefore, a device used for gene sequencing has a relatively large market space.

From the first-generation of the gene sequencing in 1977 to now, the gene sequencing technology has achieved considerable development. For example, the gene sequencing technology includes a first-generation sanger sequencing technology, a second-generation high throughput sequencing technology, a third-generation single molecule sequencing technology, and a fourth-generation nanopore sequencing technology, and a current mainstream sequencing technology is still dominated by the second-generation high throughput sequencing technology.

The second-generation high throughout sequencing technology mainly includes the technology of sequencing by synthesis of Illumina, an ion semiconductor sequencing technology of Thermo Fisher, a linking sequencing technology and a pyrosequencing technology of Roche and so on, wherein Illimina occupies more than 70% market share depending on its advantages of highest throughput and a relatively long read length.

A common gene sequencing technology modifies the bases with different fluorophores, upon the bases being paired with gene fragments to be tested, the fluorophores are released; at this moment, types of the bases can be determined by detecting fluorescence colors through an optical system, thereby obtaining a sequence of the gene fragments to be tested.

### SUMMARY

At least one embodiment of the present disclosure provides a gene sequencing chip, a gene sequencing device and a gene sequencing method. The gene sequencing chip includes a first substrate, a common electrode, a second substrate and a liquid crystal layer. The first substrate is opposite to the second substrate, the liquid crystal layer is disposed between the first substrate and the second substrate. A side of the second substrate away from the first substrate comprises at least one groove hollowed in the second substrate, the at least one groove is configured to contain a sample to be tested, a bottom of each of the at least one groove close to the first substrate is provided with an ion sensitive film, the ion sensitive film is configured to sense an ion generated by a gene sequencing reaction within the groove to generate a voltage and generate an electric field with the common electrode. Therefore, the gene sequencing chip can provide a gene sequencing which is simpler and costs lower.

At least one embodiment of the present disclosure provides a gene sequencing chip, which includes a first substrate; a common electrode; a second substrate, opposite to the first substrate; a liquid crystal layer, disposed between the first substrate and the second substrate, wherein a side of the second substrate away from the first substrate comprises at least one groove hollowed in the second substrate, the at least one groove is configured to contain a sample to be test, a bottom of each of the at least one groove close to the first substrate is provided with an ion sensitive film, the ion sensitive film is configured to sense an ion generated by a gene sequencing reaction within the groove to generate a voltage and generate an electric field with the common electrode.

For example, in the gene sequencing chip provided by an embodiment of the present disclosure, the ion sensitive film comprises a hydrogen ion sensitive film.

For example, in the gene sequencing chip provided by an embodiment of the present disclosure, the at least one groove comprises a plurality of grooves, the plurality of grooves are disposed in an array on the second substrate.

For example, in the gene sequencing chip provided by an embodiment of the present disclosure, the common electrode is disposed on a side of the first substrate close to the liquid crystal layer.

For example, in the gene sequencing chip provided by an embodiment of the present disclosure, the common electrode includes a plurality of strip-shaped common electrodes, the common electrode is disposed in a same layer with the ion sensitive film, each of the plurality of strip-shaped common electrodes is disposed between two adjacent ones of the at least one groove.

For example, in the gene sequencing chip provided by an embodiment of the present disclosure, the common electrode includes a plurality of strip-shaped common electrodes, the ion sensitive film includes a plurality of strip-shaped sensitive films, the common electrode is disposed in a same layer with the ion sensitive film, the plurality of strip-shaped common electrodes and the plurality of strip-shaped sensitive films are alternately arranged at intervals at the bottom of the at least one groove.

For example, the gene sequencing chip provided by an embodiment of the present disclosure further includes: a first polarizer; and a second polarizer, the first polarizer and the second polarizer are disposed on two sides of the liquid crystal layer.

For example, the gene sequencing chip provided by an embodiment of the present disclosure further includes: a backlight source, disposed on a side of the first polarizer away from the second polarizer, or, disposed on a side of the second polarizer away from the first polarizer.

For example, in the gene sequencing chip provided by an embodiment of the present disclosure, a shape of a section of each of the at least one groove parallel to the first substrate comprises at least one of a circle and a regular polygon.

For example, in the gene sequencing chip provided by an embodiment of the present disclosure, a maximum size of a section of each of the at least one groove parallel to the first substrate has a range of 10-100 μm.

For example, the gene sequencing chip provided by an embodiment of the present disclosure further includes: a third substrate, a sample inlet, and a sample outlet, the third substrate is disposed on a side of the second substrate away from the first substrate; the third substrate includes at least one flow channel, the at least one flow channel is communicated with the at least one groove, the sample inlet and the sample outlet are disposed on the third substrate and are communicated with the flow channel.

At least one embodiment of the present disclosure provides a gene sequencing device, which includes: a gene sequencing chip; and a photosensitive device, the gene sequencing chip includes the gene sequencing chip described in any one of the above, the photosensitive device is configured to sense emergent light at a position where the at groove is located.

For example, in the gene sequencing device provided by an embodiment of the present disclosure, the photosensitive device includes a CCD image sensor.

At least one embodiment of the present disclosure provides a gene sequencing method of a gene sequencing chip, the gene sequencing chip includes the gene sequencing chip described in any one of the above, the gene sequencing method includes: introducing the sample to be tested into the groove; adding four different deoxyribonucleotide triphosphates to the groove in sequence and sensing an ion released by a base-pairing reaction through the ion sensitive film respectively; and detecting a deflection of liquid crystal and determining the deoxynucleoside triphosphates generating the base-paring reaction according to the deflection of the liquid crystal.

For example, in the gene sequencing method provided by an embodiment of the present disclosure, detecting a deflection of liquid crystals and determining the deoxynucleoside triphosphates generating the base-paring reaction according to the deflection of the liquid crystals includes: detecting the deflection of the liquid crystal by sensing polarized light passing through the liquid crystal through a photosensitive device and a polarizer.

### BRIEF DESCRIPTION OF THE DRAWINGS

In order to clearly illustrate the technical solution of embodiments of the present disclosure, the drawings of the embodiments will be briefly described in the following, it is obvious that the drawings in the description are only related to some embodiments of the present disclosure and not limited to the present disclosure.

FIG. 1 is a shematic structural diagram of a gene sequencing chip provided by an embodiment of the present disclosure;

FIG. 2a is a shematic structural diagram of another gene sequencing chip provided by an embodiment of the present disclosure;

FIG. 2b is a shematic structural diagram of another gene sequencing chip provided by an embodiment of the present disclosure;

FIG. 3 is a plan view of a groove provided by an embodiment of the present disclosure;

FIG. 4 is a shematic structural diagram of another gene sequencing chip provided by an embodiment of the present disclosure;

FIG. 5 is a plan view of a gene sequencing chip provided by an embodiment of the present disclosure;

FIG. 6 is a work schematic diagram of a gene sequencing chip provided by an embodiment of the present disclosure;

FIG. 7 is a shematic structural diagram of a gene sequencing device provided by an embodiment of the present disclosure; and

FIG. 8 is a flow diagram of a gene sequencing method provided by an embodiment of the present disclosure.

### DETAILED DESCRIPTION

In order to make objects, technical details and advantages of the embodiments of the present disclosure apparently, the technical solutions of the embodiments will be described in a clearly and fully understandable way in connection with the drawings related to the embodiments of the present disclosure. It is obvious that the described embodiments are just a part but not all of the embodiments of the present disclosure. Based on the described embodiments herein, a person having ordinary skill in the art may obtain other embodiment(s), without any inventive work, which should be within the scope of the disclosure.

Unless otherwise defined, the technical terms or scientific terms utilized herein have the same meanings as commonly understood by one of ordinary skill in the art to which the present disclosure belongs. The terms “first”, “second”, etc., which are utilized in the description and the claims of the present disclosure, are not intended to characterize any sequence, amount or importance, but distinguish various components. The terms “includes”, “comprising”, etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly.

In a common gene sequencing technology, various bases need to be modified with different fluorophores, upon the bases being paired with gene fragments to be tested, the fluorophores are released; at this moment, types of the bases can be determined by detecting fluorescence colors through an optical system, thereby obtaining a sequence of the gene fragments to be tested. In other words, the common gene sequencing technology requires not only a fluorescence labeling of the bases but also a laser light source and an optical system. Therefore, a gene sequencing system of the common gene sequencing technology is relatively complicated, and a chemical reagent utilized to perform fluorophore modification labeling on the bases are expensive, thereby increasing time and costs of the gene sequencing.

At least one embodiment of the present disclosure provides a gene sequencing chip, a gene sequencing device and a gene sequencing method. The gene sequencing chip includes a first substrate, a common electrode, a second substrate and a liquid crystal layer. The first substrate is opposite to the second substrate, the liquid crystal layer is disposed between the first substrate and the second substrate. A side of the second substrate away from the first substrate includes at least one groove hollowed in the second substrate, the groove is configured to contain a sample to be tested, a bottom of each groove close to the first substrate is provided with an ion sensitive film, the ion sensitive film can sense an ion generated by a gene sequencing reaction within the groove and generate a voltage, thereby generating an electric field with the common electrode to drive liquid crystal molecules in the liquid crystal layer to deflect. Therefore, the ion sensitive film can be utilized by the gene sequencing chip to sense the ion (for example, a hydrogen ion) generated in the base-pairing reaction and generate a voltage, and generate an electric field controlling a deflection of the liquid crystal molecules in the liquid crystal layer, thereby determining whether the base-pairing reaction occurs by utilizing a liquid crystal optical switch technology, and then the gene sequencing can be achieved. Besides, the gene sequencing technology utilizing the gene sequencing chip does not need to perform a fluorescence labeling on various bases and does not require a laser light source and an optical system, therefore, the gene sequencing technology utilizing the gene sequencing chip has a simpler system and a lower cost.

Hereafter, the gene sequencing chip, the gene sequencing device and the gene sequencing method provided by embodiments of the present disclosure will be described in detail with reference to the accompanying drawings.

An embodiment of the present disclosure provides a gene sequencing chip. FIG. 1 is a gene sequencing chip according to the present embodiment; as illustrated in FIG. 1, the gene sequencing chip includes a first substrate 110, a common electrode 120, a second substrate 130 and a liquid crystal layer 140. The first substrate 110 is opposite to the second substrate 130, the liquid crystal layer 140 is disposed between the first substrate 110 and the second substrate 130. A side of the second substrate 130 away from the first substrate 110 includes at least one groove 136 hollowed in the second substrate 130, the groove 136 can be configured to contain a sample to be tested and perform a gene sequencing to the sample to be tested; a bottom of the groove 136 close to the first substrate 110 is provided with an ion sensitive film 132, the ion sensitive film 132 can sense an ion generated by a gene sequencing reaction within the groove 136, for example, a base-pairing reaction, and generate a voltage, so that the ion sensitive film 132 can generate an electric field with the common electrode 120 so as to control liquid crystal molecules in the liquid crystal layer 140 located on a side of the second substrate 130 close to the first substrate 110 to deflect.

In the gene sequencing chip provided by the present embodiment, the ion sensitive film is utilized to sense the ion (for example, the hydrogen iron) generated by the gene sequencing reaction, such as the base-pairing reaction, within the groove 136 and generate the voltage, such as an Nernst voltage, and generate the electric field (for example, the electric field is generated by the ion sensitive film and the common electrode) that controls the deflection of the liquid crystal molecules in the liquid crystal layer, thereby determining whether the base-pairing reaction occurs by utilizing a liquid crystal optical switch technology, and then achieving the gene sequencing. For example, as illustrated in FIG. 1, the sample to be tested is containd in the groove 136, and four different deoxyribonucleotide triphosphates 220 are added to the groove 136 in sequence. As illustrated in a left-hand groove in FIG. 1, in a case that the sample to be tested generate the base-pairing reaction with the currently added deoxyribonucleotide triphosphates, the ion sensitive film senses the ion (for example, the hydrogen iron) released by the base-pairing reaction and can generate the voltage, so as to form the electric field with the common electrode and control the liquid crystal molecules in the liquid crystal layer at a position of the groove to deflect; as illustrated in a right-hand groove in FIG. 1, in a case that the sample to be tested generate no base-pairing reaction with the currently added deoxyribonucleotide triphosphates, the ion sensitive film does not generate the voltage, the liquid crystal molecules in the liquid crystal layer at the position of the groove do not deflect. At the moment, it can be determined whether a polarization direction or a rotation direction of the light passing through the liquid crystal layer at the position of the groove is changed (for example, polarized light is irradiated to a side of the liquid crystal layer, and a polarization analyzer and the light sensitive device on the other side of the liquid crystal layer are utilized to detect whether there is light passing through the liquid crystal layer) to determine whether there is a voltage on the ion sensitive film, thereby determining whether the sample to be tested generates the base-pairing reaction with the currently added deoxyribonucleotide triphosphates and achieving the gene sequencing. It should be noted that, the gene sequencing technology utilizing the gene sequencing chip does not need to perform a fluorescence labeling on various bases and does not require a laser light source and an optical system, therefore, the gene sequencing technology utilizing the gene sequencing chip has a simpler system and a lower cost. It should be noted that, because the base-pairing reaction of a single sample to be tested and the deoxyribonucleotide triphosphates releases fewer ions, the sample to be tested can be replicated to generate a plurality of base-pairing reactions simultaneously, so that the iron sensitive film can sense and generate the voltage.

For example, the first substrate can include a glass substrate, a plastic substrate, or other transparent substrate to facilitate light transmission.

For example, the common electrode can be a transparent metal electrode, for example, an indium tin oxide (ITO) electrode. Certainly, the embodiment of the present disclosure includes but is not limited thereto, the common electrode can also be an opaque electrode, and the common electrode can be provided with a plurality of openings to achieve light transmission.

For example, the groove can be formed by etching the second substrate. Certainly, the embodiment of the present disclosure includes but is not limited thereto, the groove can also be formed by other methods.

For example, in the gene sequencing chip provided by an example of the present embodiment, a shape of a section of the groove parallel to the first substrate includes at least one of a circle and a regular polygon. Certainly, the embodiment of the present disclosure includes but is not limited thereto.

For example, in the gene sequencing chip provided by an example of the present embodiment, a maximum size of the section of the groove parallel to the first substrate has a range of 10-100 μm. It should be noted that, upon the section of the groove parallel to the first substrate being the circle, the maximum size is a diameter of the circle; upon the section of the groove being the regular polygon, the maximum size is a diagonal line of the regular polygon.

For example, in the gene sequencing chip provided by an example of the present embodiment, the ion sensitive film can include a hydrogen ion sensitive film. It should be noted that, an electric potential of the hydrogen ion sensitive film can change in response to a hydrogen ion. For example, the hydrogen ion sensitive film can utilize a hydrogen ion recognition material fixed on the hydrogen ion sensitive film, for example, a silicon nitride (Si₃N₄), to bind a hydrogen ion selectively, so that a film electric potential or a film current can be changed. Certainly, the embodiment of the present disclosure includes but is not limited thereto, the ion sensitive film can be other ion sensitive film according to an actual situation.

For example, the hydrogen sensitive film is transparent, so as to be convenient for observing a transmission situation of the polarized light.

For example, a material of the hydrogen ion sensitive film includes an organic material or an inorganic material. For example, the material of the hydrogen ion sensitive film can be selected from one or more of silicon nitride (SiNx), lithium glass, silicon dioxide (SiO₂) and aluminum oxide (Al₂O₃).

For example, in the gene sequencing chip provided by an example of the present embodiment, as illustrated in FIG. 1, the common electrode 120 is disposed on a side of the first substrate 110 close to the liquid crystal layer 140. Therefore, in a case that the ion sensitive film 132 generates the voltage, the ion sensitive film 132 can generate the electric field perpendicular to the liquid crystal layer 140 with the common electrode 120, so that the liquid crystal molecules in the liquid crystal layer 140 can be driven to deflect. It should be noted that, the common electrode 120 illustrated in FIG. 1 is disposed on an entire surface of the first substrate 110, so that a process of patterning the common electrode 120 can be reduced. However, the embodiment of the present disclosure includes but is not limited thereto, the common electrode can also be disposed corresponding to the ion sensitive film. That is, the common electrode is only disposed on the first substrate at a position where the groove is located.

For example, in the gene sequencing chip provided by an example of the present embodiment, as illustrated in FIG. 1, the gene sequencing chip further includes a sealant 190, which is disposed between the first substrate 110 and the second substrate 120, and located in a peripheral area of the first substrate 110 to seal the liquid crystal layer 140 between the first substrate 110 and the second substrate 130.

For example, in the gene sequencing chip provided by an example of the present embodiment, the at least one groove includes a plurality of grooves, the plurality of grooves are disposed in an array on the second substrate. Therefore, a plurality of samples to be tested can be detected at the same time by disposing the plurality of grooves, thereby greatly improving efficiency of the gene sequencing. In addition, it is easy to number the plurality of grooves by arranging the plurality of grooves in an array on the second substrate.

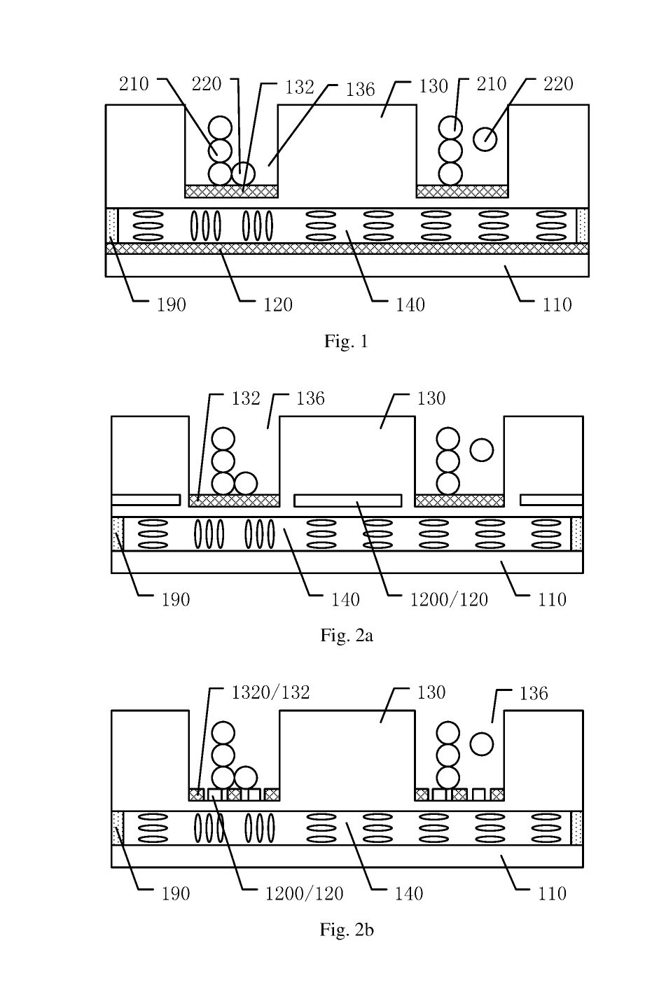

An embodiment of the present disclosure further provides a gene sequencing chip. FIG. 2a illustrates a gene sequencing chip according to the present embodiment. As illustrated in FIG. 2a, what is different from the abovementioned embodiments is that the common electrode 120 includes a plurality of strip-shaped common electrodes 1200, the common electrode 120 is disposed in a same layer with the ion sensitive film 132, each of the plurality of strip-shaped common electrodes 1200 is disposed between two adjacent ones of the grooves 136. Therefore, in a case that the ion sensitive film generates the voltage, the strip-shaped common electrodes disposed between the two adjacent ones can generate a transverse electric field with the ion sensitive film, so that the liquid crystal molecules in the liquid crystal layer can be driven to deflect. At this moment, it can be determined whether the polarization direction or the rotation direction of the light passing through the liquid crystal layer at the position of the groove is changed (for example, polarized light is irradiated to a side of the liquid crystal layer, and the polarization analyzer and the light sensitive device disposed on the other side of the liquid crystal layer are utilized to detect whether there is light passing through the liquid crystal layer) to determine whether there is the voltage on the ion sensitive film, thereby determining whether the sample to be tested generates the base-pairing reaction with the currently added deoxyribonucleotide triphosphates, so as to achieve the gene sequencing.

For example, FIG. 2b illustrates a gene sequencing chip according to the present embodiment. As illustrated in FIG. 2b, the common electrode 120 includes a plurality of strip-shaped common electrodes 1200, the ion sensitive film 132 includes a plurality of strip-shaped sensitive films 1320, the common electrode 120 is disposed in a same layer with the ion sensitive film 132, the plurality of strip-shaped common electrodes 1200 and the plurality of strip-shaped sensitive films 1320 are alternately arranged at intervals at the bottom of the grooves 136. Therefore, in a case that the ion sensitive film generates the voltage, the strip-shaped common electrodes can generate a transverse electric field with the strip-shaped sensitive films, so that the liquid crystal molecules in the liquid crystal layer can be driven to deflect. At this moment, it can be determined whether the polarization direction or the rotation direction of the light passing through the liquid crystal layer at the position of the grooves is changed (for example, polarized light is irradiated to a side of the liquid crystal layer, and the analyzer and the light sensitive device on the other side of the liquid crystal layer are utilized to detect whether there is light passing through the liquid crystal layer) to determine whether there is the voltage on the ion sensitive film, thereby determining whether the sample to be tested generates the base-pairing reaction with the currently added deoxyribonucleotide triphosphates, so as to achieve the gene sequencing.

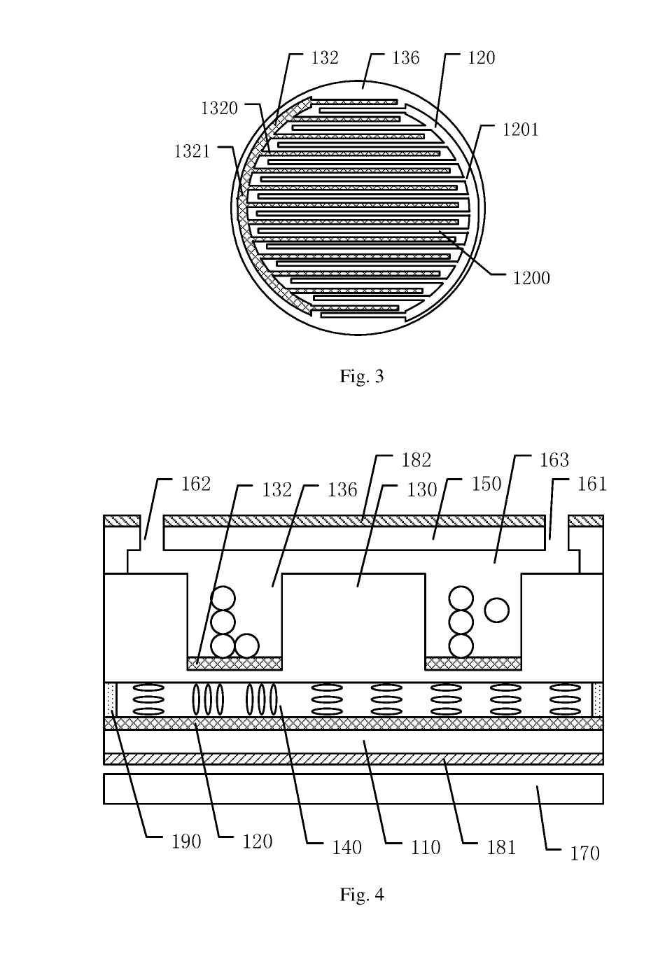

For example, FIG. 3 is a plan view of a groove of a gene sequencing chip according to the present embodiment. As illustrated in FIG. 3, in the gene sequencing chip provided by an example of the present embodiment, the common electrode 120 includes a plurality of strip-shaped common electrodes 1200, the plurality of strip-shaped common electrodes 1200 are connected with each other through a common electrode connection part 1201. The ion sensitive film 132 includes a plurality of strip-shaped sensitive film 1320, the plurality of strip-shaped common electrodes 1320 are connected with each other through an ion sensing film connection part 1321. The common electrode 120 is disposed in a same layer with the ion sensitive film 132, the strip-shaped common electrodes 1200 and the strip-shaped sensitive films 1320 are alternately arranged at intervals at the bottom of the grooves 136. Certainly, the embodiment of the present disclosure includes but is not limited thereto, the common electrode and ion sensitive film can be other shapes in a case that they are disposed in the same layer, as long as the stripe-shaped common electrodes and the strip-shaped sensitive films can generate a transverse electric field to drive the liquid crystal molecules in the liquid crystal layer to deflect.

An embodiment of the present disclosure further provides a gene sequencing chip. FIG. 4 illustrates a gene sequencing chip according to the present embodiment. As illustrated in FIG. 4, the gene sequencing chip further includes a first polarizer 181 and a second polarizer 182. The first polarizer 181 and the second polarizer 182 are disposed on two sides of the liquid crystal layer 140, a transmission axis of the first polarizer 181 is perpendicular to a transmission axis of the second polarizer 182, or, a rotation direction of the first polarizer 181 is opposite to a rotation direction of the second polarizer 182. It should be noted that, in a case that the first polarizer and the second polarizer are linear polarizers, the transmission axis of the first polarizer is perpendicular to the transmission axis of the second polarizer; in a case that the first polarizer and the second polarizer are circular polarizers or elliptical polarizers, the rotation direction of the first polarizer is opposite to the rotation direction of the second polarizer.

In the gene sequencing chip provided by the present embodiment, because the first polarizer and the second polarizer are disposed on two sides of the liquid crystal layer, and the transmission axis of the first polarizer is perpendicular to the transmission axis of the second polarizer, or, the rotation direction of the first polarizer is opposite to the rotation direction of the second polarizer, in a case that the liquid crystal molecules in the liquid crystal do not deflect, light incident from one side of the liquid crystal layer cannot exit from the other side of the liquid crystal layer. However, in a case that the sample to be tested in the grooves generates the base-pairing reaction with the currently added deoxyribonucleotide triphosphates, the ion sensitive film senses the ion (for example, the hydrogen iron) released by the base-pairing reaction and can generate the voltage, so as to form the electric field with the common electrode and control the liquid crystal molecules in the liquid crystal layer at the position where the groove is located to deflect, the light incident from one side of the liquid crystal layer can exit from the other side of the liquid crystal layer. Therefore, it can be determined that whether the sample to be tested in the grooves generates the base-pairing reaction with the currently added deoxyribonucleotide triphosphates by observing or detecting whether there is light passing through the other side of the liquid crystal layer. Therefore, the gene sequencing technology utilizing the gene sequencing chip is easy to be operated and costs lower.

It should be noted that, the embodiment of the present disclosure includes but is not limited thereto, in the gene sequencing chip provided by the present embodiment, the transmission axis of the first polarizer can be the same as the transmission axis of the second polarizer, in a case that the liquid crystal molecules in the liquid crystal layer do not deflect, the light incident from one side of the liquid crystal layer can exit from the other side of the liquid crystal layer. However, in a case that the sample to be tested in the grooves has the base-pairing reaction with the currently added deoxyribonucleotide triphosphates, the ion sensitive film senses the ion (for example, the hydrogen iron) released by the base-pairing reaction and can generate the voltage, so as to form the electric field with the common electrode and control that upon the liquid crystal molecules on the liquid crystal layer at the position of the grooves deflecting, at the position of the grooves, the light incident from one side of the liquid crystal layer cannot exit from the other side of the liquid crystal layer. Therefore, it can be determined that whether the sample to be tested in the grooves has the base-pairing reaction with the currently added deoxyribonucleotide triphosphates by observing or detecting whether there is light passing through the other side of the liquid crystal layer.

For example, in the gene sequencing chip provided by an example of the present embodiment, the gene sequencing chip further includes a backlight source 170, the backlight source 170 can be disposed on a side of the first polarizer 181 away from the second polarizer 182; or, the backlight source 170 can also be disposed on a side of the second polarizer 182 away from the first polarizer 181. For example, as illustrated in FIG. 4, the backlight source 170 is disposed on a side of the first polarizer 181 away from the second polarizer 182. Therefore, the backlight source can be integrated into the gene sequencing chip to extend a utility range of the gene detection substrate.

For example, in the gene sequencing chip provided by an example of the present embodiment, as illustrated in FIG. 4, the gene sequencing chip can further include a third substrate 150, which is disposed on a side of the second substrate 130 away from the first substrate 110. The third substrate 150 includes at least one flow channel 163, the flow channel 163 is communicated with the groove 136. Thus, the third substrate can protect the groove so as to provide a relatively stable reaction environment. In addition, four different deoxynucleoside triphosphates can also be added to the plurality of grooves through the flow channel at the same time.

For example, in the gene sequencing chip provided by an example of the present embodiment, as illustrated in FIG. 4, the gene sequencing chip further includes a sample inlet 161 and a sample outlet 162, the sample inlet 161 and the sample outlet 162 are disposed on the third substrate 150 and are connected with the flow channel 163. Thus, the four different deoxynucleoside triphosphates or detergents can be added through the sample inlet, and the four different deoxynucleoside triphosphates or the detergents can be discharged through the sample outlet.

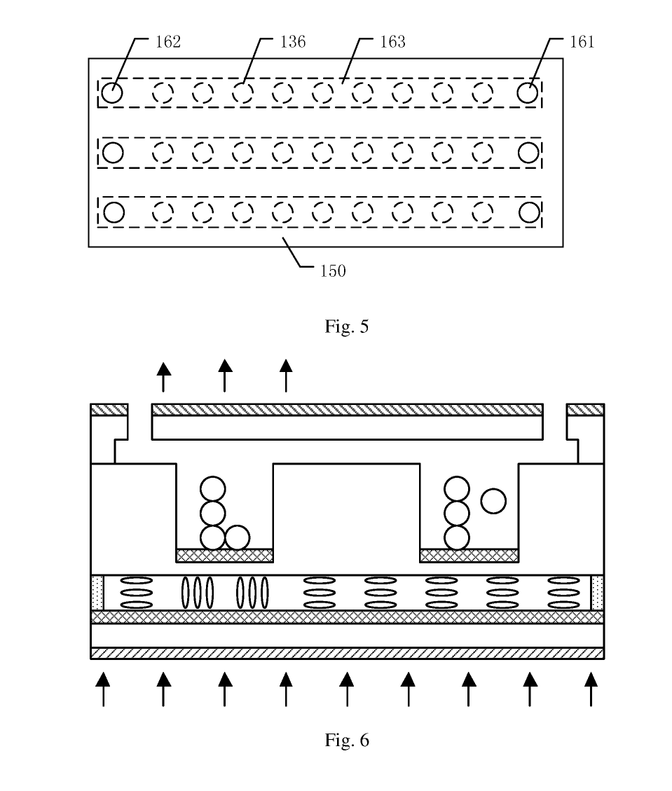

FIG. 5 illustrates a plan view of a gene sequencing chip according to the present embodiment. As illustrated in FIG. 5, the at least one groove 136 includes a plurality of grooves 136, the plurality of grooves 136 are disposed in an array.

For example, as illustrated in FIG. 5, the third substrate 150 includes a plurality of flow channels 163, which are respectively corresponding to rows of the plurality of grooves 136 disposed in an array, and each of the flow channels 163 is communicated with at least one sample inlet 161 and at least one sample outlet 162.

FIG. 6 is a work schematic diagram of a gene sequencing chip provided by an embodiment of the present disclosure. As illustrated in FIG. 6, the sample to be tested is introduced into the groove, and the four different deoxyribonucleotide triphosphates are added to the groove in sequence. As illustrated in a left-hand groove in FIG. 6, in a case that the sample to be tested generates the base-pairing reaction with the currently added deoxyribonucleotide triphosphates, the ion sensitive film senses the ion (for example, the hydrogen iron) released by the base-pairing reaction and can generate the voltage, so as to form the electric field with the common electrode and control the liquid crystal molecules in the liquid crystal layer at the position of the groove to deflect; as illustrated in a right-hand groove in FIG. 6, in a case that the sample to be tested generates no base-pairing reaction with the currently added deoxyribonucleotide triphosphates, the ion sensitive film does not generate the voltage, the liquid crystal molecules in the liquid crystal layer at the position of the groove do not deflect. At this moment, the light incident from one side of the gene sequencing chip can exit from the gene sequencing chip at a position of the left-hand groove in FIG. 6, and the light incident from one side of the gene sequencing chip cannot exit from the gene sequencing chip at other positions in FIG. 6. Thus, it can be determined that whether the sample to be tested in the grooves generates the base-pairing reaction with the currently added deoxyribonucleotide triphosphates by observing or detecting whether there is light passing through the other side of the gene sequencing chip at a position of each of the grooves. Types of current bases of the sample to be tested in the groove can be obtained by recording species of the currently added deoxyribonucleotide triphosphates. A base sequence of the sample to be tested can be obtained by a plurality of abovementioned processes.

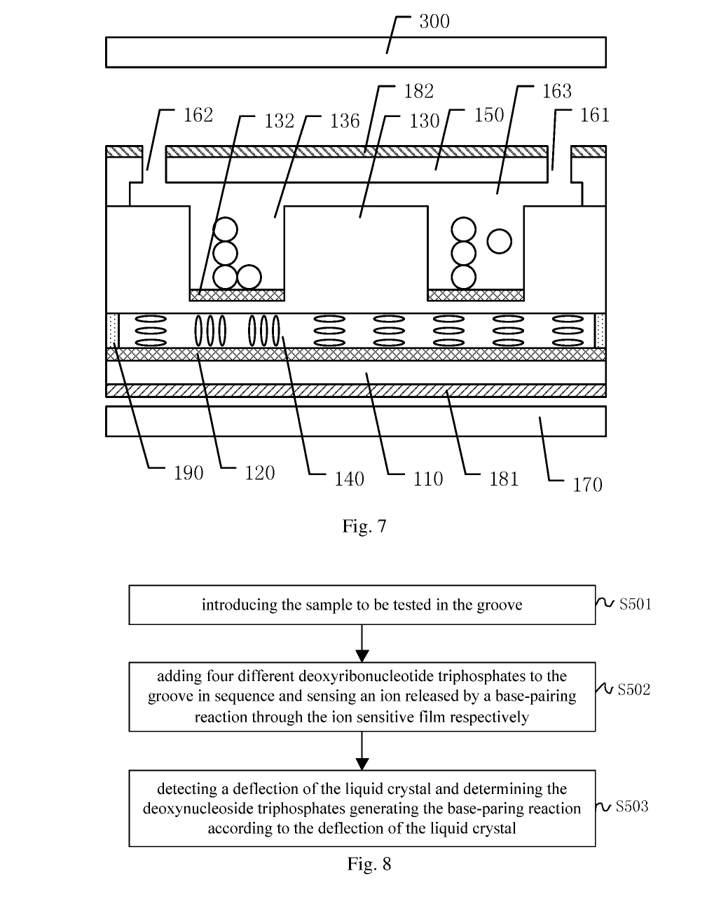

An embodiment of the present disclosure provides a gene sequencing device. FIG. 7 illustrates a gene sequencing device according to the present embodiment. As illustrated in FIG. 7, the gene sequencing device includes the gene sequencing chip according to any of the abovementioned embodiments. The gene sequencing device further includes a photosensitive device, the photosensitive device is configured to sense emergent light at a position where the at least one groove is located. It should be noted that, the emergent light at the position where the abovementioned groove is located refers emergent light of ambient light or light emitted from the backlight source incident from one side of the gene sequencing chip and exciting through the position of the groove of the gene sequencing chip. Thus, the photosensitive device can be utilized to determine whether the light incident from one side of the gene sequencing chip is exited from the position where the groove is located.

For example, in the gene sequencing device provided by an example of the present embodiment, the photosensitive device can include a CCD image sensor. Because the CCD image sensor has a high sensitivity, and can convert an optical signal into an analog current signal, it is easy to analyze by utilizing a computer.

An embodiment of the present disclosure provides a gene sequencing method of a gene sequencing chip. The gene sequencing chip utilizes the gene sequencing chip described in any one of the abovementioned embodiments. FIG. 8 illustrates a gene sequencing method according to the present embodiment. As illustrated in FIG. 8, the gene sequencing method includes steps of S501 to S503.

Step S501: introducing the sample to be tested in the at least one groove.

Step S502: adding four different deoxyribonucleotide triphosphates to the at least one groove in sequence and sensing an ion released by a base-pairing reaction through the ion sensitive film respectively.

For example, the ion sensitive film can control the deflection of liquid crystal by forming at least one of a transverse electric field, a longitudinal electric field, and a multi-dimensional electric field with the common electrode.

Step S503: detecting a deflection of the liquid crystal and determining the deoxynucleoside triphosphates generating the base-paring reaction according to the deflection of the liquid crystals.

In the gene sequencing method provided by the present embodiment, in a case that the currently added deoxyribonucleotide triphosphates generate the base-pairing reaction with the sample to be tested, the ion sensitive film can sense the ion released by the base-pairing reaction and can generate a sensing voltage, the voltage generates the electric field with the common electrode, so as to determine that the currently added deoxyribonucleotide triphosphates have the base-pairing reaction with the sample to be tested by the deflection of the liquid crystals. In a case that the currently added deoxyribonucleotide triphosphates have no base-pairing reaction with the sample to be tested, the ion sensitive film does not generate the voltage, the liquid crystals do not deflect, so as to determine the currently added deoxyribonucleotide triphosphates generate no base-pairing reaction with the sample to be tested by a case that the liquid crystals have no deflection. Types of current bases in the sample to be tested in the groove can be obtained by recording species of the currently added deoxyribonucleotide triphosphates. A base sequence of the sample to be tested can be obtained by a plurality of abovementioned processes.

For example, the four deoxyribonucleotide triphosphates including different bases (for example, in the four deoxyribonucleotide triphosphates including different bases, and four bases can be cytosine, guanine, adenine, and thymine, or cytosine, guanine, adenine, and uracil) are added to the groove in sequence, the four deoxyribonucleotide triphosphates including different bases are contacted with the sample to be tested, for example a DNA fragment, in the groove in sequence. In a case that bases in the sample to be tested have the base-pairing reaction with the currently added deoxyribonucleotide triphosphates, the ion is released, for example the hydrogen ion. At this moment, the ion sensitive film can sense the ion released by the base-pairing reaction and can generate a sensing voltage, the voltage generates the electric field with the common electrode, so as to determine that the currently added deoxyribonucleotide triphosphates have the base-pairing reaction with the sample to be tested by a case that the liquid crystals have a deflection; the base sequence of the sample to be tested can be obtained by a plurality of abovementioned processes. The gene sequencing method can achieve the gene sequencing without performing a fluorescence labeling with different colors on the four different bases, which can simplify the process of the gene sequencing; and the system utilizing the gene sequencing method is simpler and lower in cost, which is beneficial to the promotion and utilization of the gene sequencing technology.

For example, the deoxynucleoside triphosphates include a reversible terminator deoxynucleoside triphosphate, the gene sequencing method further includes: cleaning the reversible terminator deoxynucleoside triphosphate added to the groove, and adding a mercapto reagent. After completing a base type detection of a previous position of the sample to be tested (for example, the DNA fragment), the reversible terminator deoxynucleoside triphosphate added to the groove need to be cleaned, and then the mercapto reagent is added. It should be noted that unlike an ordinary deoxynucleoside triphosphate, a 3-end of the reversible terminator deoxynucleoside triphosphate is linked to an azide group, which does not form a phosphodiester bond during a DNA synthesis process, and thus the DNA synthesis process can be interrupted. Assuming that the mercapto reagent is added, the azide group will break, and a hydroxyl group can be formed at an original position. A base type detection of a subsequent location of the sample to be tested can be performed after adding the mercapto reagent, the detection method is the same as the abovementioned method, which is not repeated herein.

For example, in a case that the sample to be tested is the DNA fragment, the abovementioned the reversible terminator deoxynucleoside triphosphate can include a reversible terminator deoxy adenine triphosphate, a reversible terminator deoxy thymine triphosphate, a reversible terminator deoxy cytosine triphosphate, and a reversible terminator deoxy guanine triphosphate. In a case that the reacted deoxynucleoside triphosphate added to the groove is the deoxy adenine triphosphate, and then the base in the sample to be tested (for example, the DNA fragment) is the thymine; in a case that the reacted deoxynucleoside triphosphate added to the groove is the deoxy thymine triphosphate, and then the base in the sample to be tested (for example, the DNA fragment) is the adenine; in a case that the reacted deoxynucleoside triphosphate added to the groove is the deoxy cytosine triphosphate, and then the base in the sample to be tested (for example, the DNA fragment) is the guanine; in a case that the reacted deoxynucleoside triphosphate added to the groove is the deoxy guanine triphosphate, and then the base in the sample to be tested (for example, the DNA fragment) is the cytosine.

For example, in the gene sequencing method provided by an example of the present embodiment, the deflection of the liquid crystals can be determined by sensing a deflection of polarized light passing through the liquid crystal by the photosensitive device and the polarizer. For example, in a case that a polarization direction of the polarizer is perpendicular to a polarization direction of the polarized light, or, a rotation direction of the polarizer is opposite to a rotation direction of the polarized light, assuming that the liquid crystals do not deflect, and then the photosensitive device cannot sense the polarized light passing through the liquid crystal; assuming that the liquid crystals deflect, the polarization direction of the polarized light changes due to the deflection of the liquid crystals, and then the photosensitive device can sense the polarized light passing through the liquid crystal. It should be noted that, the abovementioned polarized light can be generated by an additionally provided polarizer.

For example, in the gene sequencing method provided by an example of the present embodiment, the method of introducing the sample to be tested in the at least one groove can include: increasing the sample to be tested to form a plurality of identical samples to be tested; and introducing the plurality of identical samples to the grooves. Because the base-pairing reaction of a single sample to be tested with the deoxyribonucleoside triphosphate releases fewer ions, the sample to be tested can be increased to generate a plurality of base-pairing reactions simultaneously, so that the ion sensitive film can sense and generate the voltage.

The following points should to be explained:

1) The drawings of at least one embodiment of the present disclosure only relate to the structure in the embodiment of the present disclosure, and other structures may be referenced to the usual design.

2) In the absence of conflict, the features of the same embodiment and the different embodiments ban be combined with each other.

The foregoing is only the embodiments of the present invention and not intended to limit the scope of protection of the present invention, alternations or replacements which can be easily envisaged by any skilled person being familiar with the present technical field shall fall into the protection scope of the present disclosure. Thus, the protection scope of the present disclosure should be based on the protection scope of the claims.

1. A gene sequencing chip, comprising:
a first substrate;
a common electrode;
a second substrate, opposite to the first substrate;
a liquid crystal layer, disposed between the first substrate and the second substrate,
wherein a side of the second substrate away from the first substrate comprises at least one groove hollowed in the second substrate, the at least one groove is configured to contain a sample to be tested, a bottom of each of the at least one groove close to the first substrate is provided with an ion sensitive film, the ion sensitive film is configured to sense an ion generated by a gene sequencing reaction within the groove to generate a voltage and generate an electric field with the common electrode.

2. The gene sequencing chip according to claim 1, wherein the ion sensitive film comprises a hydrogen ion sensitive film.

3. The gene sequencing chip according to claim 1, wherein the at least one groove comprises a plurality of grooves, the plurality of grooves are disposed in an array on the second substrate.

4. The gene sequencing chip according to claim 1, wherein the common electrode is disposed on a side of the first substrate close to the liquid crystal layer.

5. The gene sequencing chip according to claim 3, wherein the common electrode comprises a plurality of strip-shaped common electrodes, the common electrode is disposed in a same layer with the ion sensitive film, each of the plurality of strip-shaped common electrodes is disposed between two adjacent ones of the at least one groove.

6. The gene sequencing chip according to claim 1, wherein the common electrode comprises a plurality of strip-shaped common electrodes, the ion sensitive film comprises a plurality of strip-shaped sensitive patterns, the common electrode is disposed in a same layer with the ion sensitive film, the plurality of strip-shaped common electrodes and the plurality of strip-shaped sensitive patterns are alternately arranged at intervals at the bottom of the at least one groove.

7. The gene sequencing chip according to claim 1, further comprising:
a first polarizer; and
a second polarizer,
wherein the first polarizer and the second polarizer are disposed on two sides of the liquid crystal layer.

8. The gene sequencing chip according to claim 7, further comprising:
a backlight source, disposed on a side of the first polarizer away from the second polarizer, or, disposed on a side of the second polarizer away from the first polarizer.

9. The gene sequencing chip according to claim 1, wherein the ion sensitive film is transparent.

10. The gene sequencing chip according to claim 1, further comprising: a sealant, disposed between the first substrate and the second substrate, and located in a peripheral area of the first substrate to seal the liquid crystal layer between the first substrate and the second substrate.

11. The gene sequencing chip according to claim 1, wherein a shape of a section of each of the at least one groove parallel to the first substrate comprises at least one of a circle and a regular polygon.

12. The gene sequencing chip according to claim 1, wherein a maximum size of a section of each of the at least one groove parallel to the first substrate has a range of 10-100 μm.

13. The gene sequencing chip according to claim 1, further comprising:
a third substrate, disposed on a side of the second substrate away from the first substrate;
a sample inlet; and
a sample outlet,
wherein the third substrate comprises at least one flow channel, the at least one flow channel is communicated with the at least one groove, the sample inlet and the sample outlet are disposed on the third substrate and are communicated with the flow channel.

14. A gene sequencing device, comprising:
a gene sequencing chip; and
a photosensitive device,
wherein the gene sequencing chip comprises the gene sequencing chip according to claim 1, the photosensitive device is configured to sense emergent light at a position where the groove is located.

15. The gene sequencing device according to claim 14, wherein the photosensitive device comprises a CCD image sensor.

16. A gene sequencing method of a gene sequencing chip, the gene sequencing chip comprises the gene sequencing chip according to claim 1, the gene sequencing method comprises:
introducing the sample to be tested into the groove;
adding four different deoxyribonucleotide triphosphates into the groove in sequence and sensing an ion released by a base-pairing reaction through the ion sensitive film respectively; and
detecting a deflection of liquid crystal and determining the deoxynucleoside triphosphates generating a base-paring reaction according to the deflection of the liquid crystal.

17. The gene sequencing method according to claim 16, wherein detecting a deflection of liquid crystals and determining the deoxynucleoside triphosphates generating the base-paring reaction according to the deflection of the liquid crystals comprises:
detecting the deflection of the liquid crystal by sensing polarized light passing through the liquid crystals through a photosensitive device and a polarizer.

18. The gene sequencing method according to claim 16, wherein the deoxynucleoside triphosphates comprise a reversible terminator deoxynucleoside triphosphate, the gene sequencing method further comprises: cleaning the reversible terminator deoxynucleoside triphosphate added in the groove, and adding a mercapto reagent.

19. The gene sequencing chip according to claim 2, wherein the common electrode comprises a plurality of strip-shaped common electrodes, the common electrode is disposed in a same layer with the ion sensitive film, each of the plurality of strip-shaped common electrodes is disposed between two adjacent ones of the at least one groove.
